(19) United States
(12) Patent Application Publication
(10) Pub. No.: US 2010/0056429 A1
(43) Pub. Date: Mar. 4, 2010

(54) TREATMENT OF RESPIRATORY CHAIN DISORDERS USING COMPOUNDS HAVING ERYTHROPOIETIN OR THROMBOPOIETIN ACTIVITY

(76) Inventors: Guy M. Miller, Monte Sereno, CA (US); William D. Shrader, Belmont, CA (US)

Correspondence Address: MORRISON & FOERSTER LLP, 755 PAGE MILL RD, PALO ALTO, CA, 94304-1018, US

(21) Appl. No.: 12/522,912

(22) PCT Filed: Jan. 9, 2008
(86) PCT No.: PCT/US08/00353

§ 371 Date: Oct. 20, 2009

Related U.S. Application Data
(60) Provisional application No. 60/879,943, filed on Jan. 10, 2007.

(51) Int. Cl. A61K 38/22; A61P 11/00

(52) U.S. Cl. 514/8

(57) ABSTRACT

Methods of treating mitochondrial respiratory chain disorders using compounds having erythropoietin activity or thrombopoietin activity are disclosed. Indicators for assessing the efficacy of treatment are discussed.

### CROSS REFERENCE TO RELATED APPLICATIONS

This application claims priority benefit of U.S. Provisional Application No. 60/879,943, filed Jan. 10, 2007. The entire contents of that application are hereby incorporated by reference herein.

### TECHNICAL FIELD

The application discloses methods of treating mitochondrial respiratory chain disorders, such as respiratory chain protein disorders, using compounds having erythropoietin activity or thrombopoietin activity.

### BACKGROUND

Mitochondria are organelles in eukaryotic cells, popularly referred to as the “powerhouse” of the cell. The molecule adenosine triphosphate (ATP) functions as an energy “currency” or energy carrier in the cell, and eukaryotic cells derive the majority of their ATP from biochemical processes carried out by mitochondria. These biochemical processes include the citric acid cycle (the tricarboxylic acid cycle, or Kreb's cycle), which generates reduced nicotinamide adenine dinucleotide (NADH+H⁺) from oxidized nicotinamide adenine dinucleotide (NAD⁺), and oxidative phosphorylation, during which NADH+H⁺ is oxidized back to NAD⁺. (The citric acid cycle also reduces flavin adenine dinucleotide, or FAD, to FADH₂; FADH₂ also participates in oxidative phosphorylation.)

The respiratory chain is located in the inner mitochondrial membrane and consists of five multimeric protein complexes: Complex I; (approximately 46 subunits), Complex II (approximately 4 subunits), Complex II (approximately 11 subunits), Complex IV (approximately 13 subunits) and Complex V (approximately 16 units). The respiratory chain also requires two small electron carriers, ubiquinone (coenzyme Q10) and cytochrome c. ATP synthesis involves two coordinated processes. First electrons (actually hydrogen ions derived from NADH and FADH₂ in intermediary metabolism) are transported horizontally from complexes I and II to coenzyme Q to Complex III to cytochrome c to Complex IV, and ultimately to the final electron acceptor, molecular oxygen, thereby producing water. At the same time, protons are pumped “vertically” across the mitochondrial inner membrane (i.e., from the matrix to the inter membrane space) by complexes I, II, II and IV. ATP is generated by the influx of these protons back into the mitochondrial matrix through complex V (mitochondrial ATP synthase) (Di Mauro, S., Mitochondrial Medicine, 7-9 (2006). The energy released as these electrons traverse the complexes is used to generate a proton gradient across the inner membrane of the mitochondrion, which creates an electrochemical potential across the inner membrane.

Complex I (NADH dehydrogenase, also called NADH:ubiquinone oxidoreductase) removes two electrons from NADH and transfers them to a lipid-soluble carrier, ubiquinone. The reduced product, ubiquinol, is free to diffuse within the membrane. At the same time, Complex I moves four protons (H⁺) across the membrane, producing a proton gradient. Complex I is one of the main sites at which premature electron leakage to oxygen occurs, thus being one of main sites of production of a harmful free radical called superoxide.

Complex II (succinate dehydrogenase) is not a proton pump. It serves to funnel additional electrons into the quinone pool by removing electrons from succinate and transferring them (via FAD) to the quinone pool. Complex II consists of four protein subunits: SDHA, SDHB, SDHC, and SDHD. Other electron donors (e.g., fatty acids and glycerol 3-phosphate) also funnel electrons into the quinone pool (via FAD), again without producing a proton gradient.

Complex III (cytochrome bc₁ complex) removes two electrons from QH₂ and transfers them to two molecules of cytochrome c, a water-soluble electron carrier located within the intermembrane space. At the same time, it moves two protons across the membrane, producing a proton gradient (in total 4 protons: 2 protons are translocated and 2 protons are released from ubiquinol). When electron transfer is hindered (by a high membrane potential, point mutations or respiratory inhibitors such as antimycin A), Complex III may leak electrons to oxygen resulting in the formation of superoxide, a highly-toxic species, which is thought to contribute to the pathology of a number of diseases, including aging.

Complex IV (cytochrome c oxidase) removes four electrons from four molecules of cytochrome c and transfers them to molecular oxygen (O₂), producing two molecules of water (H₂O). At the same time, it moves four protons across the membrane, producing a proton gradient.

Complex V (mitochondrial ATP synthetase) which is not directly associated with Complexes I, II, III and IV uses the energy stored by the electrochemical gradient to convert ADP into ATP.

The citric acid cycle and oxidative phosphorylation are preceded by glycolysis, in which a molecule of glucose is broken down into two molecules of pyruvate, with net generation of two molecules of ATP per molecule of glucose. The pyruvate molecules then enter the mitochondria, where they are completely oxidized to CO₂ and H₂O via oxidative phosphorylation (the overall process is known as aerobic respiration). The complete oxidation of the two pyruvate molecules to carbon dioxide and water yields about at least 28-29 molecules of ATP, in addition to the 2 molecules of ATP generated by transforming glucose into two pyruvate molecules. If oxygen is not available, the pyruvate molecule does not enter the mitochondria, but rather is converted to lactate, in the process of anaerobic respiration.

The overall net yield per molecule of glucose is thus approximately at least 30-31 ATP molecules. ATP is used to power, directly or indirectly, almost every other biochemical reaction in the cell. Thus, the extra (approximately) at least 28 or 29 molecules of ATP contributed by oxidative phosphorylation during aerobic respiration are critical to the proper functioning of the cell. Lack of oxygen prevents aerobic respiration and will result in eventual death of almost all aerobic organisms; a few organisms, such as yeast, are able to survive using either aerobic or anaerobic respiration.

When cells in an organism are temporarily deprived of oxygen, anaerobic respiration is utilized until oxygen again becomes available or the cell dies. The pyruvate generated during glycolysis is converted to lactate during anaerobic respiration. The buildup of lactic acid is believed to be responsible for muscle fatigue during intense periods of activity, when oxygen cannot be supplied to the muscle cells. When oxygen again becomes available, the lactate is converted back into pyruvate for use in oxidative phosphorylation.

Genetic defects which affect cellular energy states can lead to severe disease states. One such disease linked to dysfunction in a respiratory chain is Leber's Hereditary Optic Neuropathy (LHON). The disease is characterized by blindness which occurs on average between 27 and 34 years of age (World-Wide-Web address .ncbi.nlm.nih.gov/entrez/dispomim.cgi?id=535000); blindness can develop in both eyes simultaneously, or sequentially (one eye will develop blindness, followed by the other eye shortly thereafter). Other symptoms may also occur, such as cardiac abnormalities and neurological complications.

Yet another devastating syndrome resulting from a respiratory chain disorder is mitochondrial myopathy, encephalopathy, lactacidosis, and stroke (MELAS). The disease can manifest itself in infants, children, or young adults. Strokes, accompanied by vomiting and seizures, are one of the most serious symptoms; it is postulated that the metabolic impairment of mitochondria in certain areas of the brain is responsible for cell death and neurological lesions, rather than the impairment of blood flow as occurs in ischemic stroke. Other severe complications, including neurological symptoms, are often present, and elevated levels of lactic acid in the blood occur.

Yet another devastating syndrome resulting from a respiratory chain disorder is Myoclonus Epilepsy Associated with Ragged-Red Fibers (MERRF) syndrome, one of a group of rare muscular disorders that are called mitochondrial encephalomyopathies. Mitochondrial encephalomyopathies are disorders in which a defect in the genetic material arises from a part of the cell structure that releases energy (mitochondria). This can cause a dysfunction of the brain and muscles (encephalomyopathies). The mitochondrial defect as well as “ragged-red fibers” (an abnormality of tissue when viewed under a microscope) are always present. The most characteristic symptom of MERRF syndrome is myoclonic seizures that are usually sudden, brief, jerking spasms that can affect the limbs or the entire body. Impairment of the ability to coordinate movements (ataxia), as well as an abnormal accumulation of lactic acid in the blood (lactic acidosis) may also be present in affected individuals. Difficulty speaking (dysarthria), optic atrophy, short stature, hearing loss, dementia, and involuntary jerking of the eyes (nystagmus) may also occur.

Yet another devastating syndrome resulting from a respiratory chain disorder is Pearson Syndrome, characterized by symptoms associated with bone marrow and pancreas dysfunction. It is caused by single mitochondrial DNA deletions. Those who survive infancy usually develop Kearns-Sayre Syndrome

Yet another devastating syndrome resulting from a respiratory chain disorder is Co-Enzyme Q10 Deficiency, the symptoms of which include encephalomyopathy, mental retardation, exercise intolerance, ragged-red fibers, and recurrent myoglobin in the urine.

Yet another syndrome resulting from a respiratory chain disorder is Complex I Deficiency or NADH dehydrogenase NADH-CoQ reductase deficiency, the symptoms of which are classified by three major forms: (1) fatal infantile multisystem disorder, characterized by developmental delay, muscle weakness, heart disease, congenital lactic acidosis, and respiratory failure; (2) myopathy beginning in childhood or in adult life, manifesting as exercise intolerance or weakness; and (3) mitochondrial encephalomyopathy (including MELAS), which may begin in childhood or adult life and consists of variable combinations of symptoms and signs, including ophthalmoplegia, seizures, dementia, ataxia, hearing loss, pigmentary retinopathy, sensory neuropathy, and uncontrollable movements.

Yet another syndrome resulting from a respiratory chain disorder is Complex II Deficiency or succinate dehydrogenase deficiency, the symptoms of which include encephalomyopathy and various manifestations, including failure to thrive, developmental delay, hypotonia, lethargy, respiratory failure, ataxia, myoclonus and lactic acidosis.

Yet another devastating syndrome resulting from a respiratory chain disorder is Complex III Deficiency or Ubiquinone-cytochrome c oxidoreductase deficiency, symptoms of which are categorized in four major forms: (1) fatal infantile encephalomyopathy, congenital lactic acidosis, hypotonia, dystrophic posturing, seizures, and coma; (2) encephalomyopathies of later onset (childhood to adult life): various combinations of weakness, short stature, ataxia, dementia, hearing loss, sensory neuropathy, pigmentary retinopathy, and pyramidal signs. (3) myopathy, with exercise intolerance evolving into fixed weakness; and (4) infantile histiocytoid cardiomyopathy.

Yet another syndrome resulting from a respiratory chain disorder is Complex IV Deficiency or Cytochrome c oxidase deficiency, caused by a defect in Complex IV of the respiratory chain, the symptoms of which can be categorized in two major forms: (1) encephalomyopathy, where normal development is typically seen for the first 6 to 12 months of life, followed by developmental regression, ataxia, lactic acidosis, optic atrophy, ophthalmoplegia, nystagmus, dystonia, pyramidal signs, respiratory problems and frequent seizures; and (2) myopathy, which has two main variants: (a) Fatal infantile myopathy, which may begin soon after birth, accompanied by hypotonia, weakness, lactic acidosis, ragged-red fibers, respiratory failure, and kidney problems: and (b) Benign infantile myopathy, which may begin soon after birth, accompanied by hypotonia, weakness, lactic acidosis, ragged-red fibers, respiratory problems, but which may (if the child survives) be followed by spontaneous improvement.

Yet another syndrome resulting from a respiratory chain disorder is Complex V Deficiency or ATP synthase deficiency which includes symptoms such as slow, progressive myopathy.

Yet another syndrome resulting from a respiratory chain disorder is CPEO or Chronic Progressive External Ophthalmoplegia Syndrome which includes symptoms such as visual myopathy, retinitis pigmentosa, or dysfunction of the central nervous system.

Another disease involving respiratory chain proteins is Kearns-Sayre Syndrome (KSS). KSS is characterized by a triad of features including: (1) typical onset in persons younger than age 20 years; (2) chronic, progressive, external ophthalmoplegia; and (3) pigmentary degeneration of the retina. In addition, KSS may include cardiac conduction defects, cerebellar ataxia, and raised cerebrospinal fluid (CSF) protein levels (e.g., >100 mg/dL). Additional features associated with KSS may include myopathy, dystonia, endocrine abnormalities (e.g., diabetes, growth retardation or short stature, and hypoparathyroidism), bilateral sensorineural deafness, dementia, cataracts, and proximal renal tubular acidosis. Thus, KSS may affect many organ systems.

Friedreich's ataxia (FRDA or FA) is an autosomal recessive neurodegenerative and cardiodegenerative disorder caused by decreased levels of the protein frataxin. There are several hypotheses for the role of frataxin in mitochondria: frataxin is believed to be important for the assembly of iron-sulfur clusters in mitochondrial respiratory-chain complexes; it may play a role in iron transport; it may play a role in iron storage; it may stimulate oxidative phosphorylation; and it may have anti-oxidant function (see Sturm et al., J. Biol. Chem. 280:6701 (2005)). Frataxin itself, however, does not appear to be incorporated into any of mitochondrial complexes I-V. Estimates of the prevalence of FRDA in the United States range from 1 in every 22,000-29,000 people (see World-Wide-Web address .nlm.nih.gov/medlineplus/ency/article/001411.htm) to 1 in 50,000 people (World-Wide-Web address.umc-cares.org/health_info/ADAM/Articles/001411.asp). The disease causes the progressive loss of voluntary motor coordination (ataxia) and cardiac complications. Symptoms typically begin in childhood, and the disease progressively worsens as the patient grows older; patients eventually become wheelchair-bound due to motor disabilities. Erythropoietin has been proposed for the treatment of FRDA; see International Patent Application No. WO 2006/050819 and Sturm et al., Eur. J. Clin. Invest. 35:711 (2005). MitoQ has been proposed for treating FRDA (see U.S. Patent Application Publication No. 2005/0043553). The compound idebenone has also been proposed for treatment of FRDA. While the clinical effects of idebenone have been relatively modest, the complications of mitochondrial diseases can be so severe that even marginally useful therapies are preferable to the untreated course of the disease.

Leigh's disease is a rare inherited neurometabolic disorder characterized by degeneration of the central nervous system. Leigh's disease can be caused by mutations in mitochondrial DNA or by deficiencies of pyruvate dehydrogenase. Symptoms of Leigh's disease usually begin between the ages of 3 months to 2 years and progress rapidly. In most children, the first signs may be poor sucking ability and loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function. Heart problems may also occur. In rare cases, Leigh's disease can begin during late adolescence or early adulthood and progress more slowly.

Very few treatments are available for patients suffering from these diseases. Administration of coenzyme Q10 (CoQ10) and vitamin supplements has shown only transient beneficial effects in individual cases. Accordingly, there is a serious and unmet need for effective treatments of diseases involving respiratory chain disorders.

### DISCLOSURE OF THE INVENTION

The invention embraces methods of treating respiratory chain disorders, that is, a disorder which results in the decreased utilization of oxygen by a mitochondrion, cell, tissue, or individual, due to a defect or disorder in a component contained in the mitochondrial respiratory chain.

In one embodiment, the invention embraces methods of treating respiratory chain protein disorders, that is, a disorder which results in the decreased utilization of oxygen by a mitochondrion, cell, tissue, or individual, due to a defect or disorder in a protein contained in the mitochondrial respiratory chain.

In one embodiment, the invention embraces a method of treating a respiratory chain disorder, comprising administering a therapeutically effective amount of a composition comprising one or more molecules having erythropoietin (EPO) activity or thrombopoietin (TPO) activity to an individual with a respiratory chain disorder. In another embodiment, the respiratory chain disorder is a respiratory chain protein disorder. In one embodiment the composition comprises one or more molecules having erythropoietin activity. In one embodiment the composition comprises one or more molecules having thrombopoietin activity. The composition comprising one or more molecules having EPO activity can be EPO or a biosimilar, a variant, or a mutant thereof; a protein or peptide mimetic of EPO; or a small molecule mimetic of EPO. The composition comprising one or more molecules having TPO activity can be TPO or a biosimilar, a variant, or a mutant thereof; a protein or peptide mimetic of TPO; or a small molecule mimetic of TPO. In one embodiment, a composition comprising one or more molecules having EPO activity is administered. In another embodiment, a composition comprising one or more molecules having TPO activity is administered. In another embodiment, a composition comprising both one or more molecules having EPO activity and one or more molecules having TPO activity is administered.

In another embodiment, the invention embraces a method of treating a mitochondrial disease, comprising administering a therapeutically effective amount of a composition comprising one or more molecules having erythropoietin activity to an individual with a mitochondrial disease; with the proviso that the mitochondrial disease is not Friedreich's ataxia or Leigh's syndrome.

In a further embodiment, the invention embraces a method of treating a mitochondrial disease, comprising administering a therapeutically effective amount of a composition comprising one or more molecules having erythropoietin activity to an individual with a mitochondrial disease, where the mitochondrial disease is selected from defects and abnormalities in respiratory chain proteins affecting the normal functioning of the respiratory chain.

In a further embodiment, the invention embraces a method of treating a mitochondrial disease, comprising administering a therapeutically effective amount of a composition comprising one or more molecules having erythropoietin activity to an individual with a mitochondrial disease selected from Leber's hereditary optic neuropathy (LHON); mitochondrial myopathy, encephalopathy, lactacidosis, and stroke (MELAS); Kearns-Sayre syndrome (KSS); Myoclonus Epilepsy Associated with Ragged-Red Fibers (MERRF); chronic progressive external ophthalmoplegia (CPEO); Pearson syndrome; Co-Enzyme Q10 Deficiency; Complex I Deficiency; Complex II Deficiency; Complex III Deficiency; Complex IV Deficiency; Complex V Deficiency; leukodystrophy; paraganglioma; pheochromocytoma; GRACILE syndrome; and Type II diabetes arising from mutations in mitochondrial DNA.

In a further embodiment, the invention embraces a method of treating a mitochondrial disease, comprising administering a therapeutically effective amount of a composition comprising one or more molecules having erythropoietin activity to an individual with Leber's hereditary optic neuropathy (LHON).

In a further embodiment, the invention embraces a method of treating a mitochondrial disease, comprising administering a therapeutically effective amount of a composition comprising one or more molecules having erythropoietin activity to an individual with a mitochondrial disease selected from mitochondrial myopathy, encephalopathy, lactacidosis, and stroke (MELAS); and Myoclonus Epilepsy Associated with Ragged-Red Fibers (MERRF).

In a further embodiment, the invention embraces a method of treating a mitochondrial disease, comprising administering a therapeutically effective amount of a composition comprising one or more molecules having erythropoietin activity to an individual with a mitochondrial disease selected from Kearns-Sayre syndrome (KSS) and chronic progressive external ophthalmoplegia (CPEO).

In a further embodiment, the invention embraces a method of treating a mitochondrial disease, comprising administering a therapeutically effective amount of a composition comprising one or more molecules having erythropoietin activity to an individual with a mitochondrial disease selected from Complex I Deficiency; Complex II Deficiency; Complex III Deficiency; Complex IV Deficiency; and Complex V Deficiency.

In a further embodiment, the invention embraces a method of treating a mitochondrial disease, comprising administering a therapeutically effective amount of a composition comprising one or more molecules having erythropoietin activity to an individual with a mitochondrial disease selected from Co-Enzyme Q10 Deficiency.

In another embodiment, the invention embraces a method of treating a mitochondrial disease, comprising administering a therapeutically effective amount of a composition comprising one or more molecules having thrombopoietin activity to an individual with a mitochondrial disease. In a further embodiment, the mitochondrial disease is not Friedreich's ataxia or Leigh's syndrome.

In a further embodiment, the invention embraces a method of treating a mitochondrial disease, comprising administering a therapeutically effective amount of a composition comprising one or more molecules having thrombopoietin activity to an individual with a mitochondrial disease, where the mitochondrial disease is selected from defects and abnormalities in respiratory chain proteins affecting the normal functioning of the respiratory chain.

In a further embodiment, the invention embraces a method of treating a mitochondrial disease, comprising administering a therapeutically effective amount of a composition comprising one or more molecules having thrombopoietin activity to an individual with a mitochondrial disease selected from, Leber's hereditary optic neuropathy (LHON); mitochondrial myopathy, encephalopathy, lactacidosis, and stroke (MELAS); Kearns-Sayre syndrome (KSS); Myoclonus Epilepsy Associated with Ragged-Red Fibers (MERRF); chronic progressive external ophthalmoplegia (CPEO); Pearson syndrome; Co-Enzyme Q10 Deficiency; Complex I Deficiency; Complex II Deficiency; Complex III Deficiency; Complex IV Deficiency; Complex V Deficiency; leukodystrophy; paraganglioma; pheochromocytoma; GRACILE syndrome; and Type II diabetes arising from mutations in mitochondrial DNA.

In a further embodiment, the invention embraces a method of treating a mitochondrial disease, comprising administering a therapeutically effective amount of a composition comprising one or more molecules having thrombopoietin activity to an individual with Leber's hereditary optic neuropathy (LHON).

In a further embodiment, the invention embraces a method of treating a mitochondrial disease, comprising administering a therapeutically effective amount of a composition comprising one or more molecules having thrombopoietin activity to an individual with a mitochondrial disease selected from mitochondrial myopathy, encephalopathy, lactacidosis, and stroke (MELAS); and Myoclonus Epilepsy Associated with Ragged-Red Fibers (MERRF).

In a further embodiment, the invention embraces a method of treating a mitochondrial disease, comprising administering a therapeutically effective amount of a composition comprising one or more molecules having thrombopoietin activity to an individual with a mitochondrial disease selected from Kearns-Sayre syndrome (KSS) and chronic progressive external ophthalmoplegia (CPEO).

In a further embodiment, the invention embraces a method of treating a mitochondrial disease, comprising administering a therapeutically effective amount of a composition comprising one or more molecules having thrombopoietin activity to an individual with a mitochondrial disease selected from Complex I Deficiency, Complex II Deficiency, Complex III Deficiency, Complex IV Deficiency, and Complex V Deficiency.

In a further embodiment, the invention embraces a method of treating a mitochondrial disease, comprising administering a therapeutically effective amount of a composition comprising one or more molecules having erythropoietin activity to an individual with a mitochondrial disease selected from Co-Enzyme Q10 Deficiency.

In any of the foregoing methods, the therapeutically effective amount can be an amount sufficient to improve one or more energy biomarker levels, such as pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species, or exercise tolerance, to within about at least two standard deviations of normal in a subject, more preferably within about at least one standard deviation of normal in a subject, within about at least one-half standard deviation of normal, or within about at least one-quarter standard deviation of normal. When an increase in energy biomarker levels is desired for improvement, the levels or one or more energy biomarkers are increased as indicated above; when a decrease in energy biomarker levels is desired for improvement, the levels of one or more energy biomarkers are decreased as indicated above. In another embodiment of any of the foregoing methods, when an increase in the levels of one or more energy biomarkers is desirable, the therapeutically effective amount can be an amount sufficient to increase the levels of the one or more energy biomarker by about at least 10% above the subject's level of the respective one or more energy biomarkers before treatment, by about at least 20% above the subject's level of the respective one or more energy biomarkers before treatment, by about at least 30% above the subject's level of the respective one or more energy biomarkers before treatment, by about at least 40% above the subject's level of the respective one or more energy biomarkers before treatment, by about at least 50% above the subject's level of the respective one or more energy biomarkers before treatment, by about at least 75% above the subject's level of the respective one or more energy biomarkers before treatment, or by about at least 100% above the subject's level of the respective one or more energy biomarkers before treatment. In another embodiment of any of the foregoing methods, when a decrease in a level of one or more energy biomarkers is desired, the level of the one or more energy biomarkers can be decreased by about at least 10% below the subject's level of the respective one or more energy biomarkers before treatment, by about at least 20% below the subject's level of the respective one or more energy biomarkers before treatment, by about at least 30% below the subject's level of the respective one or more energy biomarkers before treatment, by about at least 40% below the subject's level of the respective one or more energy biomarkers before treatment, by about at least 50% below the subject's level of the respective one or more energy biomarkers before treatment, by about at least 75% below the subject's level of the respective one or more energy biomarkers before treatment, or by about at least 90% below the subject's level of the respective one or more energy biomarkers before treatment.

### MODES FOR CARRYING OUT THE INVENTION

By “respiratory chain disorder” is meant a disorder which results in the decreased utilization of oxygen by a mitochondrion, cell, tissue, or individual, due to a defect or disorder in a component contained in the mitochondrial respiratory chain. By “respiratory chain” is meant the components (including, but not limited to, proteins, tetrapyrroles, and cytochromes) comprising mitochondrial complex I, II, III, IV, and/or V; “respiratory chain protein” refers to the protein components of those complexes. By “respiratory chain protein disorder” is meant a disorder which results in the decreased utilization of oxygen by a mitochondrion, cell, tissue, or individual, due to a defect or disorder in a protein contained in the mitochondrial respiratory chain. Respiratory chain protein disorders are a subset of respiratory chain disorders. Thus, a disorder such as anemia, which decreases oxygen utilization by virtue of a lack of oxygen-carrying cells in the blood, would not be embraced by the term “mitochondrial chain disorder.” Similarly, a disease such as Friedreich's ataxia, which appears to arise from a defect in frataxin (a protein important for the assembly of iron-sulfur clusters in mitochondrial respiratory-chain complexes, but which protein does not form part of the respiratory chain itself), would not be considered a respiratory chain protein disorder, as the disease does not arise from a defect or disorder in a protein contained in the mitochondrial respiratory chain.

By “therapeutically effective amount” is meant an amount sufficient to provide a measurable increase in the utilization of oxygen in an individual; and/or an amount sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

By a composition (or molecule, etc.) having “erythropoietin activity” is meant any composition (or molecule, etc.) having either all of the biological activities of erythropoietin (EPO), or having at least one of the biological activities of EPO, such as the in vivo or in vitro activity of EPO that causes an increase in production of reticulocytes and/or red blood cells by bone marrow cells. Thus, molecules that lack the in vivo or in vitro activity of causing an increase in production of reticulocytes and/or red blood cells by bone marrow cells, but retain other biological activities of EPO, are also embraced by compositions or molecules having EPO activity. The composition (or molecule, etc.) should have at least about 0.1% of one or more EPO activities, or at least about 1% of one or more EPO activities, or at least about 10% of one or more EPO activities, or at least about 20% of one or more EPO activities, on a concentration basis as compared to EPO itself.

By a composition (or molecule, etc.) having “thrombopoietin activity” is meant any composition (or molecule, etc.) having either all of the biological activities of thrombopoietin (TPO), or having at least one of the biological activities of TPO, such as the in vivo or in vitro activity of TPO that causes an increase in production of megakaryocytes and/or platelets by bone marrow cells. Thus, molecules that lack the in vivo or in vitro activity of causing an increase in production of megakaryocytes and/or platelets by bone marrow cells, but retain other biological activities of TPO, are also embraced by compositions or molecules having TPO activity. The composition (or molecule, etc.) should have at least about 0.1% of one or more TPO activities, or at least about 1% of one or more TPO activities, or at least about 10% of one or more TPO activities, or at least about 20% of one or more TPO activities, on a concentration basis as compared to TPO itself.

Erythropoietin (EPO) and thrombopoietin (TPO) have been the focus of significant research activity due to their utility in treating several serious diseases. EPO is currently approved in the United States for treatment of anemia in patients with chronic renal failure undergoing dialysis (recombinant human erythropoietin is sold under the brand name Epogen®, a registered trademark of Amgen, Inc., Thousand Oaks, Calif.). EPO is also believed to be useful in treatment of various other disorders; see, e.g., International Patent Application No. WO 2006/006165, directed to using EPO for enhancing immune responses and for the treatment of certain lymphoproliferative disorders; US 2006/0094648, directed to therapeutic or prophylactic treatment of myocardial ischemia, such as due to myocardial infarction, by administering erythropoietin; or US 2005/0272634, directed to using EPO for treatment of various disorders such as hypercholesterolemia, atherosclerosis, and diabetes.

Thrombopoietin (TPO) is a glycoprotein of approximately 332 residues (Gurney et al., Blood 85:981 (1995); the cDNA encodes a polypeptide of 353 residues, with the N-terminal 21 residues comprising a signal peptide and the remaining 332 residues comprising the mature polypeptide). Thrombopoietin is also known as Mpl-ligand or megakaryocyte growth and development factor (MGDF). The 155 amino-terminal residues of TPO from the receptor binding domain, which shows 21 percent sequence identity and 46 percent homology to erythropoietin, and retains biological activity. The remaining 177 residues show no homology to any known protein (Kaushansky, New England J. Med. 339:746 (1998)).

Molecules having erythropoietin (EPO) activity include polypeptides and proteins having at least one of the biological activities of human erythropoietin. Molecules having erythropoietin activity include, but are not limited to, erythropoietin itself, recombinant human erythropoietin, erythropoietin analogs, erythropoietin biogenerics, erythropoietin biosimilars, erythropoietin isoforms, erythropoietin mimetics, erythropoietin fragments, hybrid erythropoietin proteins, mutants of any of the foregoing molecules, erythropoietins with covalent substitutions, and any of the foregoing molecules with variant glycosylation patterns, regardless of the biological activity of the same and further regardless of the method of synthesis or manufacture thereof, including but not limited to, recombinant, whether produced from cDNA or genomic DNA, synthetic, transgenic and gene activated methods. Some examples of commercially available preparations of erythropoietin include PROCRIT® (Epoetin alfa), RETACRIT™ (Epoetin zeta), EPREX®, and ERYPRO®. Other molecules with EPO activity are disclosed in EP 640619, WO 05/051327; WO 99/66054, WO 99/38890, U.S. Pat. No. 5,688,679, WO 99/11781, EP 1064951, WO 98/05363, U.S. Pat. No. 5,643,575, WO 99/05268, WO 95/05465, WO 94/12650; and WO 91/05867; the disclosures of the use of those molecules as described in the respective patent publications are hereby incorporated by reference herein. Specific examples of cell lines modified for expression of endogenous human erythropoietin are described in international patent applications WO 99/05268 and WO 94/12650.

Erythropoietin-mimetics are molecules capable of acting as EPO in binding to the EPO receptor wherein the mimetic can have little or no apparent structural similarity to native EPO. EPO mimetics are well known to those skilled in the art. Two kinds of EPO-mimetics have been described: peptides and nonpeptides. Specific examples of erythropoietin mimetics are described in U.S. Pat. No. 5,767,078 and U.S. Pat. No. 5,773,569.

Long-acting forms of EPO are also contemplated and may be preferred in some embodiments of the present invention for administration as the second or third exposure in a dosing segment. As used herein, a “long-acting EPO” includes sustained-release compositions and formulations of EPO with increased circulating half-life, typically achieved through modification such as reducing immunogenicity and/or clearance rate, and EPO encapsulated in polymer microspheres. Examples of “long-acting EPO” include, but are not limited to, conjugates of erythropoietin with polyethylene glycol (PEG) disclosed in PCT publication WO 2002049673 (Burg et al.), PEG-modified EPO disclosed in PCT publication WO 02/32957 (Nakamura et al.), conjugates of glycoproteins having erythropoietic activity and having at least one oxidized carbohydrate moiety covalently linked to a non-antigenic polymer disclosed in PCT publication WO 94/28024 (Chyi et al.), and other PEG-EPO prepared using SCM-PEG, SPA-PEG and SBA-PEG.

By “variant” is meant a modified peptide that retains its binding properties wherein the modifications include, but are not limited to, conservative substitutions in which one or more amino acids are substituted for other amino acids; deletion or addition of amino acids that have minimal influence on the binding properties or secondary structure; conjugation of a linker; and post-translation modifications such as, for example, the addition of functional groups. Conservative amino acid substitution is an amino acid substituted by an alternative amino acid of similar charge density, hydrophilicity/hydrophobicity, size, and/or configuration (e.g., Val for Ile). One such description of conservative substitutions is defined by the BLOSUM 62 substitution matrix (Henikoff and Henikoff, Proc. Natl. Acad. Sci. USA 89:10915-10919 (1992)), where positive values can indicate conservative substitutions; however, conservative substitutions are not limited to the positive-value substitutions described in that publication. Means of making such modifications are well known in the art.

By “biosimilars” is meant copies of existing biotechnological products. Biosimilars are manufactured without access to the originator's molecular clone and original cell bank, and by a different fermentation and purification process. Although biosimilars are not identical to an existing approved product, they have demonstrated “comparability” to said approved product. Biosimilars are also sometimes referred to as “follow-on biologics.” By “erythropoietin biosimilars” is meant copies of existing erythropoietin products. By “thrombopoietin biosimilars” is meant copies of existing thrombopoietin products.

Molecules having thrombopoietin (TPO) activity include polypeptides and proteins having at least one of the biological activities of human thrombopoietin. Molecules having thrombopoietin activity include, but are not limited to, thrombopoietin itself, recombinant human thrombopoietin, thrombopoietin analogs, thrombopoietin biogenerics, thrombopoietin biosimilars, thrombopoietin isoforms, thrombopoietin mimetics, thrombopoietin fragments, hybrid thrombopoietin proteins, mutants of any of the foregoing molecules, thrombopoietin with covalent substitutions and any of the foregoing molecules with variant glycosylation patterns, regardless of the biological activity of the same and further regardless of the method or synthesis of manufacture thereof, including but not limited to, recombinant, whether produced from cDNA or genomic DNA, synthetic, transgenic and gene activated methods.

Respiratory chain disorders which can be treated by administration of compositions having EPO or TPO activity include Leber's hereditary optic neuropathy (LHON); mitochondrial myopathy, encephalopathy, lactacidosis, and stroke (MELAS); Kearns-Sayre syndrome (KSS); Myoclonus Epilepsy Associated with Ragged-Red Fibers (MERRF); chronic progressive external ophthalmoplegia (CPEO); Pearson syndrome; Co-Enzyme Q10 Deficiency; Complex I Deficiency; Complex II Deficiency; Complex III Deficiency; Complex IV Deficiency; Complex V Deficiency; leukodystrophy; paraganglioma; pheochromocytoma; GRACILE syndrome; Type II diabetes arising from mutations in mitochondrial DNA; and any other disease where defects or abnormalities in respiratory chain proteins affect the normal functioning of the respiratory chain.

Formulation and Administration of Erythropoietin and Molecules with EPO Activity

Numerous formulations of erythropoietin are known in the art, such as the commercially available PROCRIT® (Epoetin alfa), RETACRIT™ (Epoetin zeta), EPREX®, and ERYPRO®. A wide variety of other formulations are also available; see, e.g., U.S. Pat. No. 4,806,524; U.S. Pat. No. 4,992,419; U.S. Pat. No. 5,376,632; U.S. Pat. No. 5,661,125; U.S. Pat. No. 6,120,761; and U.S. Pat. No. 7,129,267. Administration of erythropoietin is also well-known in the art, as described in the foregoing documents. EPO and molecules with EPO activity can be administered to a subject via parenteral administration, including, but not limited to, intravenous, intramuscular, subcutaneous, intraperitoneal, intracerebral, intraventricular, intracerebroventricular, intrathecal, intracisternal, intraspinal and perispinal administration. EPO can also be delivered continuously or semi-continuously via pump devices. EPO can also be delivered as “long-acting EPO” including sustained-release compositions and formulations of EPO with increased circulating half-life, typically achieved through modification such as reducing immunogenicity and/or clearance rate, and EPO encapsulated in polymer microspheres. The route of administration can be selected by the health care professional in accordance with known principles. When a molecule with EPO activity is administered, the formulation, dosage, and route of administration are also determined by the health care professional in accordance with known principles; the energy biomarkers described herein can be used to monitor efficacy of treatment.

Formulation and Administration of Thrombopoietin and Molecules with TPO Activity

Numerous methods of production, formulations and methods of administration of thrombopoietin are known in the art, such as those disclosed in U.S. Pat. No. 6,790,439; U.S. Pat. No. 5,744,587; U.S. Pat. No. 5,879,673; and U.S. Pat. No. 5,986,049. TPO and molecules with TPO activity can be administered to a subject via parenteral administration, including, but not limited to, intravenous, intramuscular, subcutaneous, intraperitoneal, intracerebral, intraventricular, intracerebroventricular, intrathecal, intracistemal, intraspinal and perispinal administration. TPO can also be delivered continuously or semi-continuously via pump devices. The route of administration can be selected by the health care professional in accordance with known principles. When a molecule with TPO activity is administered, the formulation, dosage, and route of administration are also determined by the health care professional in accordance with known principles; the energy biomarkers described herein can be used to monitor efficacy of treatment.

### Clinical Assessment of Respiratory Chain Disorders and Efficacy of Therapy

Several readily measurable clinical markers are used to assess the metabolic state of patients with respiratory chain disorders. These markers can also be used as indicators of the efficacy of a given therapy, as the level of a marker is moved from the pathological value to the healthy value. These clinical markers include, but are not limited to, one or more energy biomarkers such as lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; anaerobic threshold; reduced coenzyme Q (CoQ^{red}) levels; oxidized coenzyme Q (CoQ^{ox}) levels; total coenzyme Q (CoQₜₒₜ) levels; oxidized cytochrome c levels; reduced cytochrome c levels; oxidized cytochrome c/reduced cytochrome c ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; and levels of oxygen consumption (VO2), levels of carbon dioxide output (VCO2), and respiratory quotient (VCO2/VO2). Several of these clinical markers are measured routinely in exercise physiology laboratories, and provide convenient assessments of the metabolic state of a subject. In one embodiment of the invention, the level of one or more energy biomarkers in a patient suffering from a respiratory chain disorder, such as LHON, MELAS, MERFF, Co-Enzyme Q10 Deficiency, Complex I Deficiency, Complex II Deficiency, Complex III Deficiency, Complex IV Deficiency, Complex V Deficiency, or KSS, is improved to within two standard deviations of the average level in a healthy subject. In another embodiment of the invention, the level of one or more of these energy biomarkers in a patient suffering from a respiratory chain disorder, such as LHON, MELAS, MERFF, Co-Enzyme Q10 Deficiency, Complex I Deficiency, Complex II Deficiency, Complex III Deficiency, Complex IV Deficiency, Complex V Deficiency, or KSS is improved to within one standard deviation of the average level in a healthy subject. Exercise intolerance can also be used as an indicator of the efficacy of a given therapy, where an improvement in exercise tolerance (i.e., a decrease in exercise intolerance) indicates efficacy of a given therapy.

Several metabolic biomarkers have already been used to evaluate efficacy of CoQ10, and these metabolic biomarkers can be monitored as energy biomarkers for use in the methods of the current invention. Pyruvate, a product of the anaerobic metabolism of glucose, is removed by reduction to lactic acid in an anaerobic setting or by oxidative metabolism, which is dependent on a functional mitochondrial respiratory chain. Dysfunction of the respiratory chain may lead to inadequate removal of lactate and pyruvate from the circulation and elevated lactate/pyruvate ratios are observed in mitochondrial cytopathies (see Scriver, C. R., The Metabolic and Molecular Bases of Inherited Disease, 7th ed., New York: McGraw-Hill, Health Professions Division, 1995; and Munnich et al., J. Inherit. Metab. Dis. 15(4):448-55 (1992)). Blood lactate/pyruvate ratio (Chariot et al., Arch. Pathol. Lab. Med. 118(7):695-7 (1994)) is, therefore, widely used as a noninvasive test for detection of mitochondrial cytopathies (see Scriver C. R., The Metabolic and Molecular Bases of Inherited Disease, 7th ed., New York: McGraw-Hill, Health Professions Division, 1995; and Munnich et al., J. Inherit. Metab. Dis. 15(4):448-55 (1992)) and toxic mitochondrial myopathies (Chariot et al., Arthritis Rheum. 37(4):583-6 (1994)). Changes in the redox state of liver mitochondria can be investigated by measuring the arterial ketone body ratio (acetoacetate/3-hydroxybutyrate: AKBR) (Ueda et al., J. Cardiol. 29(2):95-102 (1997)). Urinary excretion of 8-hydroxy-2′-deoxyguanosine (8-OHdG) often has been used as a biomarker to assess the extent of repair of ROS-induced DNA damage in both clinical and occupational settings (Erhola et al., FEBS Lett. 409(2):287-91 (1997); Honda et al., Leuk. Res. 24(6):461-8 (2000); Pilger et al., Free Radic. Res. 35(3):273-80 (2001); Kim et al. Environ Health Perspect 112(6):666-71 (2004)).

Magnetic resonance spectroscopy (MRS) has been useful in the diagnoses of mitochondrial cytopathy by demonstrating elevations in cerebrospinal fluid (CSF) and cortical white matter lactate using proton MRS (1H-MRS) (Kaufmann et al., Neurology 62(8):1297-302 (2004)). Phosphorous MRS (31P-MRS) has been used to demonstrate low levels of cortical phosphocreatine (PCr) (Matthews et al., Ann. Neurol. 29(4):435-8 (1991)), and a delay in PCr recovery kinetics following exercise in skeletal muscle (Matthews et al., Ann. Neurol. 29(4):435-8 (1991); Barbiroli et al., J. Neurol. 242(7):472-7 (1995); Fabrizi et al., J. Neurol. Sci. 137(1):20-7 (1996)). A low skeletal muscle PCr has also been confirmed in patients with mitochondrial cytopathy by direct biochemical measurements.

Exercise testing is particularly helpful as an evaluation and screening tool in mitochondrial myopathies. One of the hallmark characteristics of mitochondrial myopathies is a reduction in maximal whole body oxygen consumption (VO2max) (Taivassalo et al., Brain 126(Pt 2):413-23 (2003)). Given that VO2max is determined by cardiac output (Qc) and peripheral oxygen extraction (arterial-venous total oxygen content) difference, some mitochondrial cytopathies affect cardiac function where delivery can be altered; however, most mitochondrial myopathies show a characteristic deficit in peripheral oxygen extraction (A-VO2 difference) and an enhanced oxygen delivery (hyperkinetic circulation) (Taivassalo et al., Brain 126(Pt 2):413-23 (2003)). This can be demonstrated by a lack of exercise induced deoxygenation of venous blood with direct AV balance measurements (Taivassalo et al., Ann. Neurol. 51(l):38-44 (2002)) and non-invasively by near infrared spectroscopy (Lynch et al., Muscle Nerve 25(5):664-73 (2002); van Beekvelt et al., Ann. Neurol. 46(4):667-70 (1999)).

Several of these energy biomarkers are discussed in more detail as follows. It should be emphasized that, while certain energy biomarkers are discussed and enumerated herein, the invention is not limited to modulation, normalization or enhancement of only these enumerated energy biomarkers.

Lactic acid (lactate) levels: Mitochondrial dysfunction typically results in abnormal levels of lactic acid, as pyruvate levels increase and pyruvate is converted to lactate to maintain capacity for glycolysis. Mitochondrial dysfunction can also result in abnormal levels of NADH+H⁺, NADPH+H⁺, NAD, or NADP, as the reduced nicotinamide adenine dinucleotides are not efficiently processed by the respiratory chain. Lactate levels can be measured by taking samples of appropriate bodily fluids such as whole blood, plasma, or cerebrospinal fluid. Using magnetic resonance, lactate levels can be measured in virtually any volume of the body desired, such as the brain.

Measurement of cerebral lactic acidosis using magnetic resonance in MELAS patients is described in Kaufmann et al., Neurology 62(8): 1297 (2004). Values of the levels of lactic acid in the lateral ventricles of the brain are presented for two mutations resulting in MELAS, A3243G and A8344G. Whole blood, plasma, and cerebrospinal fluid lactate levels can be measured by commercially available equipment such as the YSI 2300 STAT Plus Glucose & Lactate Analyzer (YSI Life Sciences, Ohio).

NAD, NADP, NADH and NADPH levels: Measurement of NAD, NADP, NADH (NADH+H⁺) or NADPH (NADPH+H⁺) can be measured by a variety of fluorescent, enzymatic, or electrochemical techniques, e.g., the electrochemical assay described in US 2005/0067303.

Oxygen consumption (vO₂ or VO2), carbon dioxide output (vCO₂ or VCO2), and respiratory quotient (VCO2/VO2): vO2 is usually measured either while resting (resting vO₂) or at maximal exercise intensity (vO₂ max). Optimally, both values will be measured. However, for severely disabled patients, measurement of vO₂ max may be impractical. Measurement of both forms of vO₂ is readily accomplished using standard equipment from a variety of vendors, e.g., Korr Medical Technologies, Inc. (Salt Lake City, Utah). VCO2 can also be readily measured, and the ratio of VCO2 to VO2 under the same conditions (VCO2/VO2, either resting or at maximal exercise intensity) provides the respiratory quotient (RQ).

Oxidized Cytochrome c, reduced Cytochrome c, and ratio of oxidized Cytochrome c to reduced Cytochrome c: Cytochrome c parameters, such as oxidized cytochrome c levels (Cyt Cₒₓ), reduced cytochrome c levels (Cyt C_{red}), and the ratio of oxidized cytochrome c/reduced cytochrome c ratio (Cyt Cₒₓ)/(Cyt C_{red}), can be measured by in vivo near infrared spectroscopy. See, e.g., Rolfe, P., “In vivo near-infrared spectroscopy,” Ann. Rev. Biomed. Eng. 2:715-54 (2000) and Strangman et al., “Non-invasive neuroimaging using near-infrared light” Biol. Psychiatry 52:679-93 (2002).

Exercise tolerance/Exercise intolerance: Exercise intolerance is defined as “the reduced ability to perform activities that involve dynamic movement of large skeletal muscles because of symptoms of dyspnea or fatigue” (Pifia et al., Circulation 107:1210 (2003)). Exercise intolerance is often accompanied by myoglobinuria, due to breakdown of muscle tissue and subsequent excretion of muscle myoglobin in the urine. Various measures of exercise intolerance can be used, such as time spent walking or running on a treadmill before exhaustion, time spent on an exercise bicycle (stationary bicycle) before exhaustion, and the like. Treatment with the compounds or methods of the invention can result in about a 10% or greater improvement in exercise tolerance (for example, about a 10% or greater increase in time to exhaustion, e.g., from 10 minutes to 11 minutes), about a 20% or greater improvement in exercise tolerance, about a 30% or greater improvement in exercise tolerance, about a 40% or greater improvement in exercise tolerance, about a 50% or greater improvement in exercise tolerance, about a 75% or greater improvement in exercise tolerance, or about a 100% or greater improvement in exercise tolerance. While exercise tolerance is not, strictly speaking, an energy biomarker, for the purposes of the invention, it can be used to evaluate therapeutic efficacy.

Similarly, tests for normal and abnormal values of pyruvic acid (pyruvate) levels, lactate/pyruvate ratio, ATP levels, anaerobic threshold, reduced coenzyme Q (COQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species are known in the art and can be used to evaluate efficacy of therapeutic intervention.

Table 1, following, illustrates the effect that various dysfunctions can have on biochemistry and energy biomarkers. It should be noted that any of the energy biomarkers listed in the table, in addition to energy biomarkers enumerated elsewhere, can also be monitored to track the efficacy of treatment. RQ=respiratory quotient; BMR=basal metabolic rate; HR (CO)=heart rate (cardiac output); T=body temperature (preferably measured as core temperature); AT=anaerobic threshold; pH=blood pH (venous and/or arterial).

TABLE 1

| Site of | Biochemical | Measurable Energy | Physical |
|---|---|---|---|
| Dysfunction | Event | Biomarker | Effect |
| Respiratory | ↑ NADH | Δ lactate, | Metabolic |
| Chain | | Δ lactate:pyruvate ratio; | dyscrasia & |
| | | and | fatigue |
| | | Δ acetoacetate:β-hydroxy | |
| | | butyrate ratio | |
| Respiratory | ↓ H⁺ gradient | Δ ATP | Organ |
| Chain | | | dependent |
| | | | dysfunction |
| Respiratory | ↓ Electron flux | Δ VO₂, RQ, BMR, ΔT, | Metabolic |
| Chain | | AT, pH | dyscrasia & |
| | | | fatigue |
| Respiratory | ↓ Cyt C_{Ox/Red} | Δ λ ~700-900 nM (Near | Exercise |
| Chain | | Infrared Spectroscopy) | intolerance |
| Respiratory | ↓ Electron flux | Δ Mixed Venous VO₂ | Metabolic |
| Chain | | | dyscrasia & |
| | | | fatigue |

Treatment of a subject afflicted by a respiratory chain disorder in accordance with the methods of the invention may result in the inducement of a reduction or alleviation of symptoms in the subject, e.g., to halt the further progression of the disorder.

Partial or complete suppression of the respiratory chain disorder can result in a lessening of the severity of one or more of the symptoms that the subject would otherwise experience. For example, partial suppression of MELAS could result in reduction in the number of stroke-like or seizure episodes suffered.

Any one or any combination of the energy biomarkers described herein provides conveniently measurable benchmarks by which to gauge the effectiveness of treatment or suppressive therapy. Additionally, other energy biomarkers are known to those skilled in the art and can be monitored to evaluate the efficacy of treatment or suppressive therapy. Again, while exercise tolerance is not, strictly speaking, an energy biomarker, for the purposes of the invention, it can be used to evaluate therapeutic efficacy, such as for the discussion below regarding increases or decreases in energy biomarkers.

When an increase in the level of one or more of the energy biomarkers is desired, the level of the energy biomarker can be increased to within about at least two standard deviations of normal in a subject, more preferably increased to within about at least one standard deviation of normal in a subject, increased to within about at least one-half standard deviation of normal, or increased to within about at least one-quarter standard deviation of normal, by treatment with a composition having EPO activity or TPO activity according to the invention. Alternatively, the level can be increased by about at least 10% above the subject's level of the respective one or more energy biomarkers before treatment, by about at least 20% above the subject's level of the respective one or more energy biomarkers before treatment, by about at least 30% above the subject's level of the respective one or more energy biomarkers before treatment, by about at least 40% above the subject's level of the respective one or more energy biomarkers before treatment, by about at least 50% above the subject's level of the respective one or more energy biomarkers before treatment, by about at least 75% above the subject's level of the respective one or more energy biomarkers before treatment, or by about at least 100% above the subject's level of the respective one or more energy biomarkers before treatment.

When a decrease in a level of one or more energy biomarkers is desired, the level of the one or more energy biomarkers can be decreased to a level within about at least two standard deviations of normal in a subject, more preferably decreased to within about at least one standard deviation of normal in a subject, decreased to within about at least one-half standard deviation of normal, or decreased to within about at least one-quarter standard deviation of normal, by treatment with a composition having EPO activity or TPO activity according to the invention. Alternatively, the level of the one or more energy biomarkers can be decreased by about at least 10% below the subject's level of the respective one or more energy biomarkers before treatment, by about at least 20% below the subject's level of the respective one or more energy biomarkers before treatment, by about at least 30% below the subject's level of the respective one or more energy biomarkers before treatment, by about at least 40% below the subject's level of the respective one or more energy biomarkers before treatment, by about at least 50% below the subject's level of the respective one or more energy biomarkers before treatment, by about at least 75% below the subject's level of the respective one or more energy biomarkers before treatment, or by about at least 90% below the subject's level of the respective one or more energy biomarkers before treatment.

### BIOLOGICAL EXAMPLES

### Example A

Screening EPO Compounds in Fibroblasts from Leber's Hereditary Optic Neuropathy Patients for Rescue from Oxidative Stress.

Primary human fibroblasts obtained from patients with Leber's Hereditary Optic Neuropathy (LHON) purchased from the Coriell Cell Repositories (Camden, N.J.; repository number GM03858) are grown in 10 cm tissue culture plates. Every third day, they are split at a 1:3 ratio. Human dermal fibroblasts from mitochondrial disease patients have been shown to be hypersensitive to inhibition of the de novo synthesis of glutathione (GSH) with L-buthionine-(S,R)-sulfoximine (BSO), a specific inhibitor of GSH synthetase (Jauslin et al., Hum. Mol. Genet. 11(24):3055 (2002)). LHON fibroblasts were stressed by addition of L-buthionine-(S,R)-sulfoximine (BSO), as described in Jauslin et al., Hum. Mol. Genet. 11 (24):3055 (2002), Jauslin et al., FASEB J. 17:1972-4 (2003), and International Patent Application WO 2004/003565, such that cellular viability of LHON but not of healthy patient fibroblasts, was decreased. Prior to stress, cells are pre-treated with EPO and cellular viability monitored. Increased cellular viability suggests that EPO affects cellular susceptibility to oxidative stress by modulating overall cellular health.

Materials:
MEM Medium 199 with Earle's Balanced Salts and Fetal Calf Serum (Invitrogen, Carlsbad, Calif.)
Basic fibroblast growth factor and epidermal growth factor (PeproTech, Rocky Hill, N.J.).
Penicillin-streptomycin-glutamine mix (Sigma, St Louis, Mo.),
L-buthionine (S,R)-sulfoximine (Sigma, St Louis, Mo.)
Insulin from bovine pancreas (Sigma, St Louis, Mo.)
Calcein AM (Anaspec, San Jose, Calif.).

Procedure:

Cell culture medium is made by combining 125 ml M199, 50 ml Fetal Calf Serum, 100 U/ml penicillin, 100 ug/ml streptomycin, 2 mM glutamine, 10 ug/ml insulin, 10 ng/ml EGF, and 10 ng/ml bFGF; MEM is added to make the volume up to 500 ml. A 10 mM BSO solution is prepared by dissolving 444 mg BSO in 200 ml of medium with subsequent filter-sterilization. During the course of the experiments, this solution is stored at +4° C.

A culture with LHON fibroblasts is started from a 1 ml vial with approximately 500,000 cells stored in liquid nitrogen. Cells are propagated in 10 cm cell culture dishes by splitting every third day in a ratio of 1:3. Once confluent, fibroblasts are harvested to yield 3,000 cells/well in a 96 well plate. The remaining cells are distributed in 10 cm cell culture plates (600,000 cells/plate) for propagation. The plates are incubated overnight at 37° C. in an atmosphere with 95% humidity and 5% CO₂ to allow attachment of the cells to the culture plate.

Plates are kept overnight in the cell culture incubator. The next day, EPO test compounds as well as 10 ul of a 300 uM BSO solution are added to the wells, resulting in a 30 uM final BSO concentration. Forty-eight hours later, plates are examined under a phase-contrast microscope to verify that the cells in the control wells are clearly dead. The medium from all plates is discarded, and the remaining liquid is removed by gently tapping the plate inversed onto a paper towel.

After 2 washes with 100 ul of PBS, 100 ul of PBS containing 1.2 uM Calcein-AM is added to each well. The plates are incubated for 30 minutes at 37° C. and fluorescence (excitation/emission wavelengths of 485 nm and 525 nm, respectively) is read on a M2 Molecular Devices fluorescence reader. Data is imported into Microsoft Excel™ and xCell Fit is used to calculate the EC₅₀ concentration for each compound.

The viability of non-BSO treated fibroblasts is set as 100%, and the viability of the BSO- and EPO-treated cells is calculated as relative to this value.

### Example B

Screening EPO Compounds in Fibroblasts from Leber 's Hereditary Optic Neuropathy Patients for Effect on Oxidative Phosphorylation.

The effect of EPO on cellular oxidative phosphorylation is assessed via measurement of oxygen consumption in growing cells. Treated cells should have the increased use of their ETC resulting in higher oxygen consumption rate as measured with Seahorse instrument, and contain higher overall ratios of ATP/ADP as measured by HPLC. Cells are grown as described above but in the presence of pyruvate, and assayed in the presence or absence of glycolysis inhibitors, such as 3BrPa, iodoacetate, fluoride, or 2-deoxyglucose. Cells with well functioning ETC should exhibit an increase in oxygen consumption concomitant with a decrease in the media acidification rate due to glycolysis. EPO is expected to enhance the increase of oxygen consumption and the decrease of glycolysis of LHON patient primary fibroblasts.

### Example C

Screening EPO Compounds in Fibroblasts from Leber's Hereditary Optic Neuropathy Patients for Up-Regulation of ETC Components

Treatment of LHON cells with EPO may result in increased cellular ETC protein content. EPO treated cells, grown as described above, are analyzed by Western blot for ETC and other regulatory protein amounts and correlated to untreated cells. Examples of such proteins include but are not limited to, aconitase, SOD, and components of Complex I, II, III, IV, and V. Increase in ETC protein content can be correlated to the improvement of mitochondrial function and oxidative phosphorylation.

### Example D

Screening EPO Compounds in Fibroblasts from MELAS Patients for Rescue from Oxidative Stress.

Compounds of the invention are tested using the screen as described in Example A, but substituting LHON cells with MELAS cells created from immortalized fibroblasts depleted of mitochondrial DNA (rho 0 cells) and mitochondria from MELAS patient fibroblasts. The cybrid cell lines are screened to obtain a homogeneous mitochondrial population bearing the mutant mitochondrial genome.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating a respiratory chain disorder, comprising:
administering a therapeutically effective amount of a composition comprising one or more molecules having erythropoietin (EPO) activity, or a composition comprising one or more molecules having thrombopoietin (TPO) activity, to an individual with a respiratory chain disorder.

2-28. (canceled)

29. The method of claim 1, wherein the composition comprises one or more molecules having erythropoietin activity.

30. The method of claim 1, wherein the composition comprises one or more molecules having thrombopoietin activity.

31. The method of claim 1, wherein the composition comprises EPO, or a biosimilar, a variant, or a mutant thereof.

32. The method of claim 1, wherein the composition comprises TPO, or a biosimilar, a variant, or a mutant thereof.

33. The method of claim 1, wherein the composition comprises a protein or peptide mimetic of EPO.

34. The method of claim 1, wherein the composition comprises a protein or peptide mimetic of TPO.

35. The method of claim 1, wherein the composition comprises a small molecule mimetic of EPO.

36. The method of claim 1, wherein the composition comprises a small molecule mimetic of TPO.

37. A method of treating a mitochondrial disease, comprising:
administering a therapeutically effective amount of a composition comprising one or more molecules having erythropoietin activity to an individual with a mitochondrial disease;
with the proviso that the mitochondrial disease is not Friedreich's ataxia or Leigh's syndrome.

38. The method of claim 37, where the mitochondrial disease is caused by defects and/or abnormalities in respiratory chain proteins affecting the normal functioning of the respiratory chain.

39. The method of claim 37, comprising:
administering a therapeutically effective amount of a composition comprising one or more molecules having erythropoietin activity to an individual with a mitochondrial disease selected from Leber's hereditary optic neuropathy (LHON); mitochondrial myopathy, encephalopathy, lactacidosis, and stroke (MELAS); Keams-Sayre syndrome (KSS) ; Myoclonus Epilepsy Associated with Ragged-Red Fibers (MERRF); chronic progressive external ophthalmoplegia (CPEO); Pearson syndrome; Co-Enzyme Q10 Deficiency; Complex I Deficiency; Complex II Deficiency; Complex III Deficiency; Complex IV Deficiency; Complex V Deficiency; leukodystrophy; paraganglioma; pheochromocytoma; GRACILE syndrome; and Type II diabetes arising from mutations in mitochondrial DNA.

40. The method of claim 37, comprising:
administering a therapeutically effective amount of a composition comprising one or more molecules having erythropoietin activity to an individual with Leber's hereditary optic neuropathy (LHON).

41. The method of claim 37, comprising:
administering a therapeutically effective amount of a composition comprising one or more molecules having erythropoietin activity to an individual with a mitochondrial disease selected from mitochondrial myopathy, encephalopathy, lactacidosis, and stroke (MELAS); and Myoclonus Epilepsy Associated with Ragged-Red Fibers (MERRF).

42. The method of claim 37, comprising:
administering a therapeutically effective amount of a composition comprising one or more molecules having erythropoietin activity to an individual with Kearns-Sayre syndrome (KSS) or chronic progressive external ophthalmoplegia (CPEO).

43. The method of claim 37, comprising:
administering a therapeutically effective amount of a composition comprising one or more molecules having erythropoietin activity to an individual with a mitochondrial disease selected from Complex I Deficiency; Complex II Deficiency; Complex III Deficiency; Complex IV Deficiency; and Complex V Deficiency.

44. The method of claim 37, comprising:
administering a therapeutically effective amount of a composition comprising one or more molecules having erythropoietin activity to an individual with the mitochondrial disease Co-Enzyme Q10 Deficiency.

45. A method of treating a mitochondrial disease, comprising:
administering a therapeutically effective amount of a composition comprising one or more molecules having thrombopoietin activity to an individual with a mitochondrial disease.

46. The method of claim 45, with the proviso that the mitochondrial disease is not Friedreich's ataxia or Leigh's syndrome.

47. The method of claim 45 where the mitochondrial disease is caused by defects and abnormalities in respiratory chain proteins affecting the normal functioning of the respiratory chain.

48. The method of claim 45, comprising:
administering a therapeutically effective amount of a composition comprising one or more molecules having thrombopoietin activity to an individual with a mitochondrial disease selected from Leber's hereditary optic neuropathy (LHON); mitochondrial myopathy, encephalopathy, lactacidosis, and stroke (MELAS); Keams-Sayre syndrome (KSS); Myoclonus Epilepsy Associated with Ragged-Red Fibers (MERRF); chronic progressive external ophthalmoplegia (CPEO); Pearson syndrome; Co-Enzyme Q10 Deficiency; Complex I Deficiency; Complex II Deficiency; Complex III Deficiency; Complex IV Deficiency; Complex V Deficiency; leukodystrophy; paraganglioma; pheochromocytoma; GRACILE syndrome; and Type II diabetes arising from mutations in mitochondrial DNA.

49. The method of claim 45, comprising:
administering a therapeutically effective amount of a composition comprising one or more molecules having thrombopoietin activity to an individual with Leber's hereditary optic neuropathy (LHON).

50. The method of claim 45, comprising:
administering a therapeutically effective amount of a composition comprising one or more molecules having thrombopoietin activity to an individual with a mitochondrial disease selected from mitochondrial myopathy, encephalopathy, lactacidosis, and stroke (MELAS); and Myoclonus Epilepsy Associated with Ragged-Red Fibers (MERRF).

51. The method of claim 45, comprising:
administering a therapeutically effective amount of a composition comprising one or more molecules having thrombopoietin activity to an individual with Kearns-Sayre syndrome (KSS) or chronic progressive external ophthalmoplegia (CPEO).

52. The method of claim 45, comprising:
administering a therapeutically effective amount of a composition comprising one or more molecules having thrombopoietin activity to an individual with a mitochondrial disease selected from Complex I Deficiency; Complex II Deficiency; Complex III Deficiency; Complex IV Deficiency; and Complex V Deficiency.

53. The method of claim 45, comprising:
administering a therapeutically effective amount of a composition comprising one or more molecules having thrombopoietin activity to an individual with the mitochondrial disease Co-Enzyme Q10 Deficiency

54. The method of claim 1, wherein the therapeutically effective amount is an amount sufficient to improve pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species, or exercise tolerance, to within about at least two standard deviations of normal in a subject.

55. The method of claim 37, wherein the therapeutically effective amount is an amount sufficient to improve pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species, or exercise tolerance, to within about at least two standard deviations of normal in a subject.

56. The method of claim 45, wherein the therapeutically effective amount is an amount sufficient to improve pyruvic acid (pyruvate) levels, lactate/pyruvate ratio, ATP levels, anaerobic threshold, reduced coenzyme Q (CoQ^{red}) levels, oxidized coenzyme Q (CoQ^{ox}) levels, total coenzyme Q (CoQ^{tot}) levels, oxidized cytochrome c levels, reduced cytochrome c levels, oxidized cytochrome c/reduced cytochrome c ratio, acetoacetate levels, β-hydroxy butyrate levels, acetoacetate/β-hydroxy butyrate ratio, 8-hydroxy-2′-deoxyguanosine (8-OHdG) levels, and levels of reactive oxygen species, or exercise tolerance, to within about at least two standard deviations of normal in a subject.
